(19) United States
(12) Patent Application Publication
(10) Pub. No.: US 2016/0262602 A1
(43) Pub. Date: Sep. 15, 2016

(54) Laparoscopic Cholecystectomy With Fluorescence Cholangiography

(71) Applicant: Steven Sounyoung Yu, Fairfax, VA (US)

(72) Inventor: Steven Sounyoung Yu, Fairfax, VA (US)

(21) Appl. No.: 15/065,855

(22) Filed: Mar. 9, 2016

Related U.S. Application Data
(60) Provisional application No. 62/130,006, filed on Mar. 9, 2015.

(51) Int. Cl. A61B 1/04; A61B 1/00

(52) U.S. Cl. CPC A61B 1/043; A61B 1/00045; A61B 1/00009

(57) ABSTRACT

Use of near-infrared fluorescence imaging in performing an endoscopic surgery, such as laparoscopic cholecystectomy. A composite video image of the surgical field (e.g. the biliary anatomy) is shown on a display screen. The composite video image combines a visible color image and a fluorescence image of the surgical field. Using various techniques, selective fluorescence imaging of a particular area of interest in the surgical field is made possible. For example, in a cholecystectomy procedure, the biliary ducts may be the particular area of interest for the selective fluorescence imaging.

### FIELD OF THE INVENTION

My invention relates to fluorescence imaging in surgery.

### BACKGROUND

Inadvertent injury to the bile ducts from anatomic misidentification is among the more serious complications of laparoscopic cholecystectomy. Real-time intraoperative imaging of the biliary anatomy by fluorescence cholangiography is a recently introduced technique that helps the surgeon to delineate the bile ducts during the laparoscopic surgery. In this technique, indocyanine green (ICG) dye is administered intravenously to the patient prior to the operation. The exogenous dye (which is a fluorophore) accumulates in the liver and is excreted through the biliary ducts. Illumination with the proper excitation light causes the dye to fluoresce with near-infrared (NIR) light. Imaging of this NIR light gives a visual outline of the bile ducts to assist the surgeon in proper anatomic identification.

FIG. 1 shows a prior art method of fluorescence cholangiography illuminating the biliary tree during laparoscopic cholecystectomy. The top left panel shows a visible color image of the surgical field as viewed through a laparoscope. The various anatomic parts are seen in their natural color, i.e. the liver, gall bladder, pancreas, duodenum, and the biliary ducts (the cystic duct and the hepatic duct, which merge into the common bile duct). When the NIR fluorescent dye is intravenously administered, it accumulates in the liver and is excreted through the biliary tract. The top right panel shows the NIR fluorescence emission image as viewed through the laparoscope (shown in lime green pseudo-color). As seen here, there is fluorescence emission detected coming from the liver and biliary ducts. The bottom panel shows the merger of the two image, i.e. the fluorescence image is overlaid the color image. As seen here, because much of the fluorescent dye is still remaining in the liver, the fluorescence emission from the liver dominates the view and makes it more difficult to contrast the biliary ducts from the liver.

### SUMMARY OF THE INVENTION

My invention relates to the use of NIR fluorescence imaging in performing an endoscopic surgery, such as laparoscopic cholecystectomy. Use of NIR fluorescence imaging in laparoscopic cholecystectomy is sometimes referred to in the literature as fluorescence cholangiography. Near-infrared (NIR) light is in the wavelength range of about 700 to about 900 nm. A NIR fluorophore is a chemical compound that absorbs NIR light of one wavelength and converts that energy to emit NIR light of a different wavelength. This process is called fluorescence emission.

With the appropriate equipment, the fluorescence from this dye can be observed intra-operatively. Moreover, this fluorescence image can be combined with a conventional color image to make a composite image in which the fluorescence image is overlaid the color image. To allow the surgeon to simultaneously view the anatomical structures in both normal color mode and fluorescence mode, imaging systems that provide real-time overlay of the NIR fluorescence video onto the conventional color video have been introduced. Examples of apparatus that can be used for fluorescence cholangiography include those described in U.S. Patent/Publication Nos. U.S. Pat. Nos. 6,821,245; 8,473,035; U.S. Pat. No. 8,498,695; US 2011/0063427; U.S. Pat. No. 8,498,695; which are incorporated by reference herein.

My invention allows for selective fluorescence imaging of a particular area of interest in the surgical field. For example, in a cholecystectomy procedure, the biliary ducts (and in particular, the common bile duct) may be the particular area of interest for the fluorescence imaging, with the fluorescence emission from the liver obscuring this view. In my invention, a composite video image of the surgical field (e.g. the biliary anatomy) is shown on a display screen. The composite video image combines a visible color image and a fluorescence image of the surgical field.

In one aspect, my invention is a method of performing an endoscopic surgery (e.g. a cholecystectomy) on a patient. In certain embodiments, the method comprises: administering a near-infrared fluorescent dye to the patient; inserting an endoscope into the patient's body (e.g. abdominal cavity) to view the surgical field (e.g. the patient's biliary anatomy);and exposing the surgical field with white light and fluorescence excitation light. The method further comprises viewing on a display screen, a composite video image of the surgical field viewed from the endoscope, in which (a) the fluorescence image is limited to only a window portion of the composite video image that is less than the full size of the composite video image; or (b) the fluorescence image is attenuated over a portion of the color image on the basis of the color of that portion of the color image; (or a combination thereof). The method further comprises performing the endoscopic surgery using the composite video image for guidance.

In some embodiments, the method further comprises selecting the size of the window portion. In some embodiments, the method further comprises selecting the position of the window portion. In some embodiments, the method further comprises selecting the shape of the window portion.

In some embodiments, the surgical procedure is a cholecystectomy and the surgical field includes the patient's biliary anatomy. In some embodiments, the step of performing the endoscopic surgery comprises viewing one or more of the biliary ducts that is shown by the fluorescence image in the composite video image to assist with dissection of the patient's gallbladder or bile duct. In some embodiments, the method further comprises selecting the size of the window portion, selecting the position of the window portion, or selecting the shape of the window portion to encompass one or more of the biliary ducts.

In some embodiments, the fluorescence image is attenuated using a target color range, wherein the fluorescence image is attenuated over portions of the color image either (i) having a color in the target color range or (ii) not having a color in the target color range. In some embodiments, the method further comprises selecting a target color range. In some cases, this may be performed by using a color range selection tool.

In another aspect, my invention is a method of providing a video image of an endoscopic surgery on a patient. In certain embodiments, the method comprises, via an endoscope that is inserted into the patient's body, receiving a color image of a surgical field; and also via the endoscope, simultaneously receiving a fluorescence image of the surgical field as viewed through the endoscope. The method further comprises combining the color image and the fluorescence image into a composite video image in which (a) the fluorescence image is limited to only a window portion of the composite video image that is less than the full size of the composite video image; or (b) the fluorescence image is attenuated over a portion of the color image on the basis of the color of that portion of the color image; (or a combination thereof). The method further comprises displaying the composite video image on a display screen.

In some embodiments, the method further comprises, in response to a user input (e.g. through the use of a touchscreen, keyboard, or mouse), changing the size of the window portion. In some embodiments, the method further comprises, in response to a user input, changing the position of the window portion. In some embodiments, the method further comprises, in response to a user input, changing the shape of the window portion.

In some embodiments, the fluorescence image is attenuated using a target color range, wherein the fluorescence image is attenuated over portions of the color image either (i) having a color in the target color range or (ii) not having a color in the target color range. In some cases, the method further comprises providing on the display screen, a color range selection tool that can be used by the user to select the target color range. In some cases, the method further comprises receiving user input from the color range selection tool to select the target color range.

In another aspect, my invention is an apparatus for producing a video image from an endoscope. In certain embodiments, the apparatus comprises an imaging system comprising one or more digital image sensors for capturing near-infrared and visible color images; wherein the imaging system receives a color image and a fluorescence image of a surgical field, and combines the color image and the fluorescence image into a composite video image in which (a) the fluorescence image is limited to only a window portion of the composite video image that is less than the full size of the composite video image; or (b) the fluorescence image is attenuated over a portion of the color image on the basis of the color of that portion of the color image; (or a combination thereof). The apparatus further comprises a display screen that receives and displays the composite video image.

In some embodiments, the apparatus further comprises a beam splitter optically coupled to the one or more digital image sensors. In some embodiments, the apparatus further comprises one or more light sources for providing illumination light and fluorescence excitation light. In some embodiments, the display screen is a touchscreen display. In some embodiments, the apparatus further comprises an endoscope that is coupled to the imaging system, the beam splitter, and the one or more light sources.

In certain embodiments, the method for performing an endoscopic surgery (e.g. a cholecystectomy) on a patient comprises: administering a near-infrared fluorescent dye to the patient; inserting an endoscope into the patient's body to view a surgical field; exposing the surgical field with illumination light and fluorescence excitation light; viewing on a display screen, a composite video image that combines a visible color image and a fluorescence image of the surgical field; selecting a fluorescence imaging condition to enhance or attenuate the fluorescence image at a first part of the surgical field relative to the fluorescence image at a second part of the surgical field; and performing the endoscopic surgery using the composite video image for guidance.

In some embodiments, the fluorescence image is generated by spectrally-resolved fluorescence imaging. In some embodiments, the fluorescence image is generated by time-resolved fluorescence imaging.

In certain embodiments, the method for providing a video image of an endoscopic surgery on a patient comprises: via an endoscope that is inserted into the patient's body, exposing a surgical field with an illumination light and an excitation light; via the endoscope, receiving a color image of the surgical field; via the endoscope, receiving a fluorescence image of the surgical field; combining the color image and the fluorescence image into a composite video image; receiving a user selection of a fluorescence imaging condition; enhancing or attenuating the fluorescence image at a first part of the surgical field relative to the fluorescence image at a second part of the surgical field; displaying the composite video image on a display screen.

In some embodiments, the fluorescence image is generated by spectrally-resolved fluorescence imaging. In some embodiments, the fluorescence image is generated by time-resolved fluorescence imaging. In some embodiments, the fluorescence imaging condition is a wavelength for the excitation light or a wavelength for detection of fluorescent light emitted from the surgical field. In some cases, the wavelength is selected by an adjustable wavelength selector.

In certain embodiments, the apparatus for producing a video image from an endoscope comprises: one or more light sources for providing illumination light and fluorescence excitation light; one or more digital image sensors for capturing near-infrared and visible color images; a user controller for (a) selecting a wavelength for the excitation light, (b) selecting a wavelength for detecting near-infrared fluorescence emission, (c) selecting a time for detecting near-infrared fluorescence emission, or (d) selecting a frequency for pulsing or modulating the excitation light; an imaging system that operates to combine the color image and the fluorescence image into a composite video image; and a display screen that receives and displays the composite video image.

In some embodiments, the imaging system generated the fluorescence image by spectrally-resolved fluorescence imaging. In some embodiments, the imaging system generated the fluorescence image by time-resolved fluorescence imaging. In some embodiments, the apparatus further comprises an adjustable wavelength selector. In some cases, the adjustable wavelength selector provides a selection that comprises at least three wavelength bands that are continuously adjacent or overlapping with each other. The multiple possible wavelength bands are within the range of 700-900 nm. In some cases, each of the wavelength bands has a width of less than 25 nm.

### BRIEF DESCRIPTION OF THE DRAWINGS

FIG. 1 shows a prior art method of fluorescence cholangiography illuminating the biliary tree during laparoscopic cholecystectomy.

FIG. 2 shows the fluorescence image overlay being limited to an oval window.

FIG. 3 shows the fluorescence image overlay over the liver being blocked.

FIG. 4A shows the user positioning a selection circle on the gallbladder. FIG. 4B shows the user positioning a selection circle on the liver.

FIG. 5 shows an example of a color range selection tool.

FIGS. 6A and 6B show a filter wheel that can be used for selecting a wavelength.

FIG. 7 shows a linear filter that can be used for selecting a wavelength.

FIGS. 8A-C show an example of how selecting the wavelength for emission detection can allow selective imaging of the biliary ducts.

FIGS. 9A-C show an example of how selecting the time for detecting fluorescence emission can allow for can allow selective imaging of the biliary ducts.

FIG. 10 shows an example apparatus of my invention.

FIG. 11 shows an example laparoscope set-up of my invention.

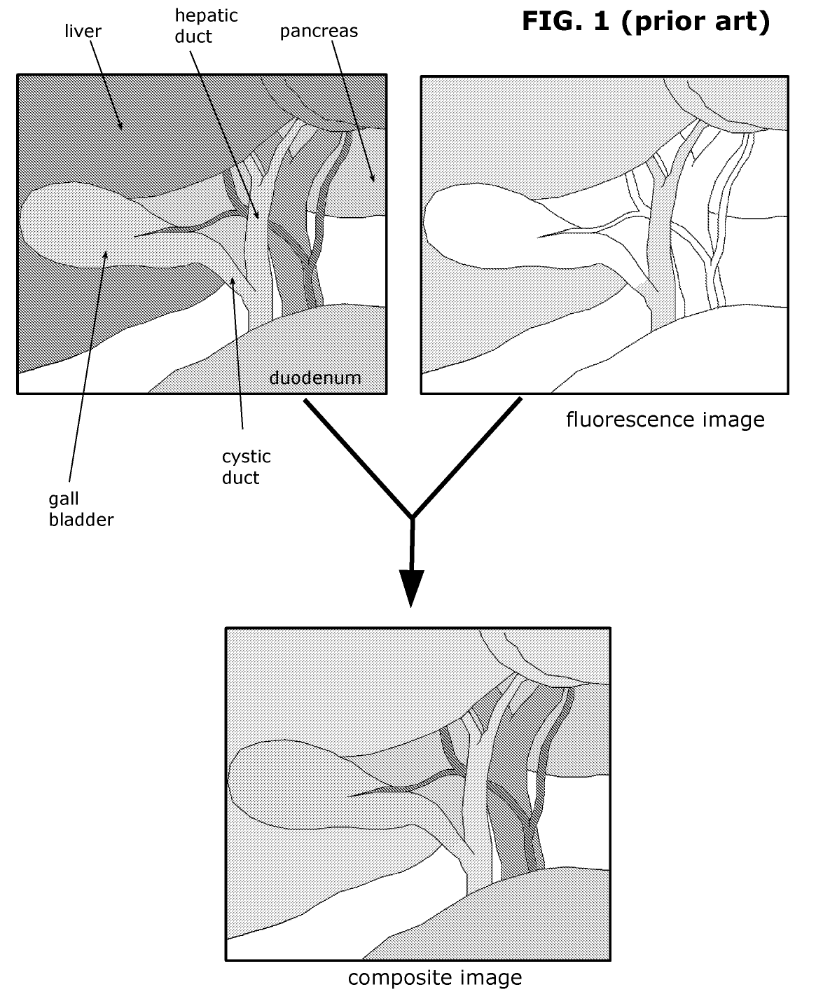

The patent or application file contains some drawings in color. Copies of this patent or patent application publication with color drawings will be provided by the Patent Office upon request and payment of the necessary fee.

### DETAILED DESCRIPTION OF THE INVENTION

In my invention, a fluorescent video image of the surgical field is composited with a color video image of the surgical field in real-time to produce a composited video image. In the composited video image, the fluorescence video image (which is in greyscale) is shown in a pseudo-color. Other terms of the art that are equivalent or encompassed by the term “compositing” include blending, overlaying, superimposing, or merging the images. The expression stating that the fluorescence image is overlaid or superimposed over the color image does not necessarily indicate any particular order of layering. Whether the fluorescence image is treated as a layer over the color image or the reverse arrangement, the same desired effect can be rendered, i.e. that the fluorescence image appears to the human user as being placed over the color image.

The image processing required to composite the color video image with the NIR fluorescence video image may be performed by any suitable image processing technique(s), including analog imaging methods (e.g. using specialized hardware), digital imaging methods, or combinations thereof. Digital compositing is commonly performed using linear blending algorithms (such as alpha blending). Other compositing methods that can be used include the compositing techniques used in Viz Engine by Vizrt or the PixelConduit tool by Lacquer. The image processing for implementing my invention may be realized in hardware and/or software.

For compositing the two video images, synchronization may be needed. For example, it is possible that the visible color images and fluorescence images may be captured at different frame rates. In such cases, synchronization of the two moving images may be accomplished using any suitable technique such as frame rate adjustment. For example, if the color video camera is capturing images at the conventional rate of thirty frames-per-second and the NIR camera is capturing images at a frame rate of fifteen frames-per-second, the two may be synchronized by reducing the frame rate of the color video images to the frame rate of the NIR video images, by using every other frame of the color video image or averaging or otherwise interpolating the color video image to a slower frame rate. Alternatively, the two may be synchronized by increasing the frame rate of the NIR video images, by holding each NIR video image frame over successive frames of the color video images or extrapolating, such as by warping the NIR video image according to changes in the color video image, or employing other known image processing techniques.

It is possible that registration or alignment of the two video images in relation to each other may be required to properly overlay the fluorescence image onto the color image. This can be accomplished by any suitable image processing technique. For example, software may be used to automatically detect common reference points within the two images in order to properly align the two images. This alignment can also be performed by spatial image registration of the fluorescence image(s) and the color images. If the color and fluorescence images are captured by a single camera (e.g. as in U.S. Pat. No. 8,498,695), then no alignment may be needed.

For effective intra-operative use, the image processing should be sufficiently fast that the composite video image can be acquired and displayed on the display screen in real-time, i.e. with any delay being sufficiently short that a surgeon experienced in endoscopic surgery would consider it acceptable for use as intra-operative guidance. This time range can be a few millisecond or shorter. Often, the pseudo-color for the grayscale NIR image is shown in lime green. However, any other pseudo-color may be used as well.

1. Fluorescence Overlay Window Portion. In some embodiments, in the composited video image, the fluorescence image overlay is limited to only a window portion of the composite video image that is less than the full size of the composite video image. In some cases, the window portion is not more than 75% of the full size of the composite video image; in some cases, not more than 60% of the full size of the composite video image. There may be one or more such window portions in the composite video image.

As used herein, “full size” of the composite video image does not necessarily mean that the composite video image takes up the entirety of the display screen. For example, the composite video image may be shown as a smaller frame within the display screen. The term “composite video image” does not necessarily mean that the entire color video image is composited with the fluorescence image. Instead, it means that at least some portion of the color video image is composited with the fluorescence image.

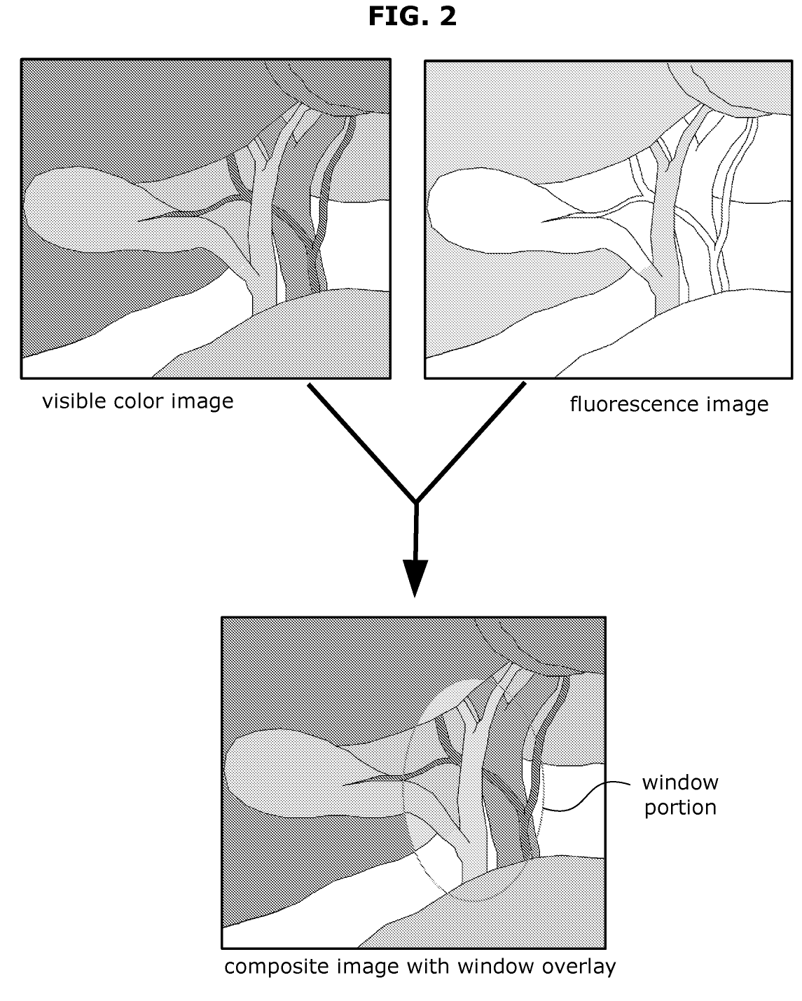

For example, FIG. 2 shows how the biliary ducts could be illuminated with fluorescence cholangiography. The top left panel shows a color image of the surgical field as viewed through a laparoscope. The top right panel shows the NIR fluorescence emission image as viewed through the laparoscope (shown in lime green pseudo-color). The bottom panel shows the merger of the two images, i.e. the fluorescence image is overlaid the color image. In this instance, the fluorescence image overlay is limited to an oval window over the color image. By limiting the fluorescence image overlay to this oval window portion of the color image, contrasting visualization of the biliary structures is improved.

In some embodiments, the user can select the size of the fluorescence image overlay window portion to make it bigger or smaller. In the context of a cholecystectomy surgery, this feature can allow the user to adjust the size of the window to better balance viewing of the fluorescence emission coming from the biliary structures, while avoid covering the liver as much as possible. In some cases, the user selects the size of the window to encompass a view of one or more of the biliary ducts.

In some embodiments, the user can select the position of the window portion to encompass the desired area. In some cases, in the context of a cholecystectomy surgery, the user may select the position of the window to encompass a view of one or more of the biliary ducts. For example, the user may move the window portion to better balance viewing of the fluorescence emission coming from the biliary structures, while avoid covering the liver as much as possible.

In some embodiments, the user can select the shape of the window (e.g. circle, oval, diamond, square, user-defined shape, etc.). In some cases, in the context of a cholecystectomy surgery, the user may select the shape of the window to encompass a view of one or more of the biliary ducts. For example, the user may find that a diamond-shaped window provides a better balance for viewing of the fluorescence emission coming from the biliary structures, while avoid covering the liver as much as possible.

2. Attenuation of Fluorescence Overlay. In some embodiments, the fluorescence image overlay is attenuated over portions of the color video image on the basis of the color of that portion of the color video image. As used herein, the term “attenuating” means eliminating or reducing the fluorescence image overlay. In the context of a cholecystectomy surgery, this feature may be useful in reducing or eliminating parts of the fluorescence image overlay that do not involve the biliary ducts such as the fluorescence emitted from the liver.

As a person skilled in this art would understand, this attenuation effect can be achieved by various means such as increasing the transparency of the fluorescence pseudo-color image overlay. For example, in alpha blending, the fluorescence pseudo-color overlay can be made more translucent in the desired areas. Whatever the means used, for that pixel or group of pixels, the fluorescence pseudo-color overlay (if any) is reduced or eliminated. In another example, the compositing step may simply be omitted over portions of the color image in which fluorescence overlay is undesired.

The attenuation can be targeted to portions of the color image (e.g. individual pixel-by-pixel, sets or groups of pixels, discrete objects or features as recognized by image processing) based on the color of that portion. For example, the liver generally has a reddish-brown hue. In this example, attenuation of the fluorescence overlay can be targeted to portions of the color image having a reddish-brown hue.

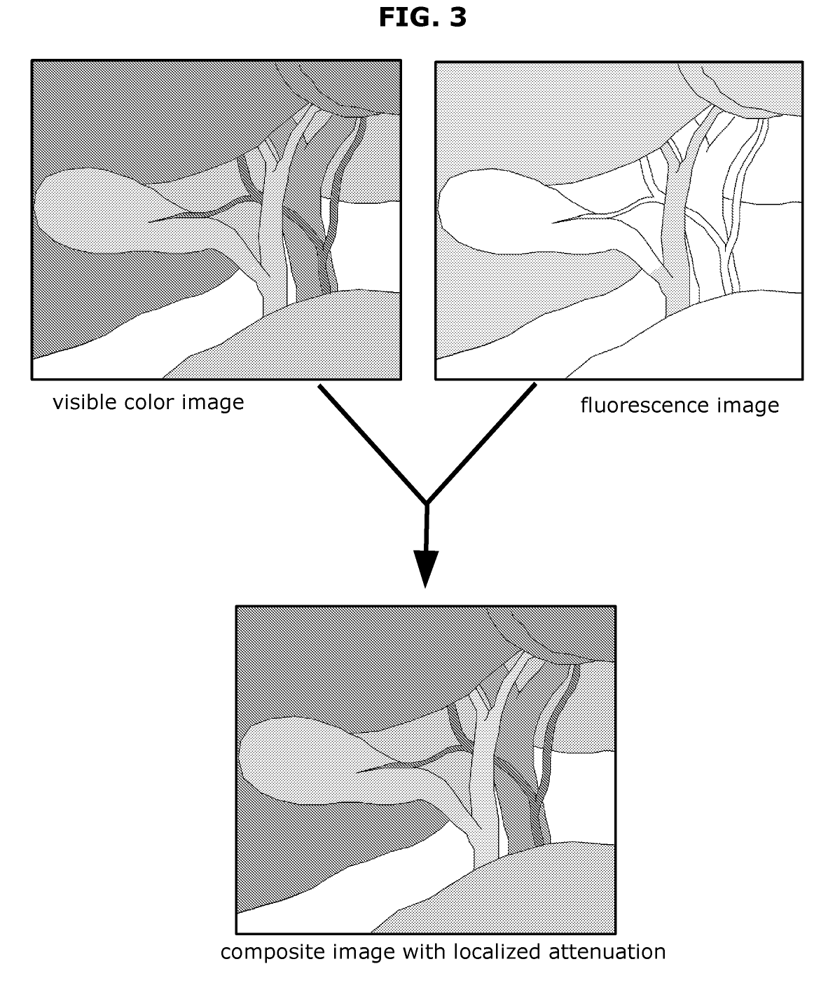

For example, FIG. 3 shows how the biliary ducts could be illuminated with fluorescence cholangiography. The top left panel shows a color image of the surgical field as viewed through a laparoscope. The top right panel shows the NIR fluorescence emission image as viewed through the laparoscope (shown in lime green pseudo-color). The bottom panel shows the merger of the two images, i.e. the fluorescence image is overlaid the color image. In this instance, the fluorescence image overlay is eliminated over the liver portion of the color video image. By blocking the fluorescence image overlay over the liver, contrasting visualization of the biliary structures is improved.

This attenuation technique can be implemented by using a target color range. The target color range can be preselected, selected by the user, or a combination thereof. This target color range can represent the range of colors over which attenuation of the fluorescence pseudo-color overlay is desired or not desired. The fluorescence pseudo-color overlay is attenuated over portions of the color image either (a) having a color in the target color range or (b) not having a color in the target color range. A target color range can be a single continuous range or a collection of multiple (two or more) discontinuous ranges (e.g.

separate domains on a color palette). The color range can be represented in any suitable dimension (e.g. on a line, a 2D color space, or 3D cylinder).

2(a). Attenuation Selection. In some embodiments, the target color range represents the range of colors over which fluorescence pseudo-color overlay is desired. The fluorescence image overlay is attenuated over portions in the color image having a color that is outside this target color range. The result is that the NIR fluorescence image overlay is permitted over parts of the image having the target color range, but attenuated over parts of the color image having a color that is outside this target color range.

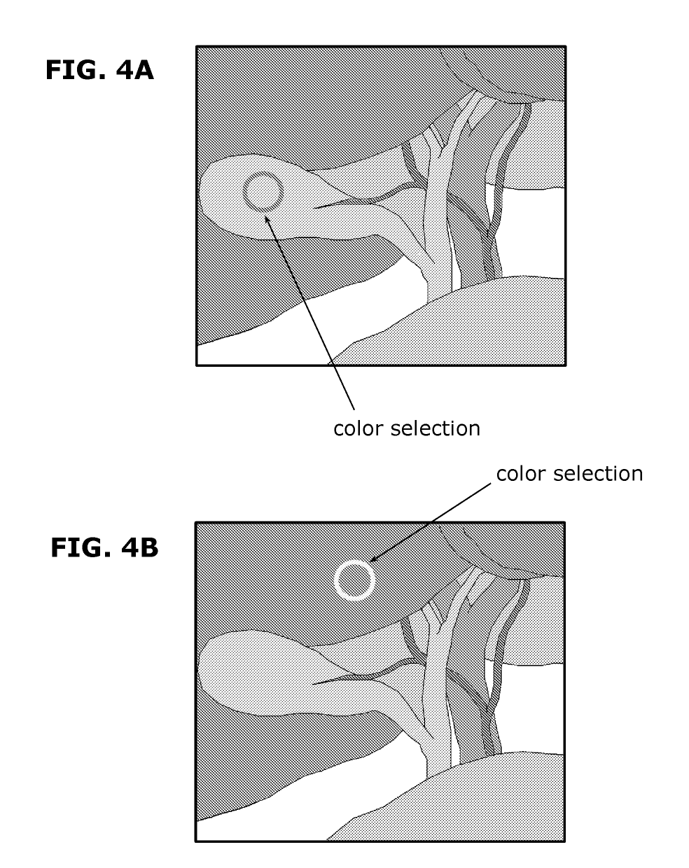

For example, the fatty tissue surrounding the biliary ducts have a generally yellow-reddish hue. As shown in FIG. 4A, this yellow-reddish range could be selected by the user positioning a selection circle to mark the desired portion of the color image (in this case, the color of the gallbladder). The colors inside the selection circle becomes the target color range in which the fluorescence pseudo-color overlay is permitted (i.e. attenuation is not desired). But for portions of the color video image having a color outside of the target color range, the fluorescence pseudo-color overlay is attenuated in the composited image. The resulting effect in the composited image is that fluorescence pseudo-color overlay is shown over the biliary ducts, but is attenuated over portions of the image outside the target color range such as the liver, which has a darker and more reddish-blue or brown color.

In some embodiments, the fluorescence image overlay is attenuated over portions in the color image having a color that is in the target color range. Thus, the target color range represents the range of colors over which attenuation of the fluorescence pseudo-color overlay is desired. For portions of the color image having colors outside of this range, the fluorescence pseudo-color overlay is not attenuated. The result is that the NIR fluorescence pseudo-color overlay is attenuated over portions of the image having the target color range, but permitted over other portions of the image.

For example, as explained above, liver tissue generally has a darker reddish-blue or brown hue. As shown in FIG. 4B, this reddish-brown range could be selected by the user positioning a selection circle to mark the desired portion of the color image (in this case, the color of the liver). The colors inside the selection circle becomes the target color range in which the fluorescence pseudo-color overlay should be attenuated. The resulting effect is that the fluorescence pseudo-color overlay occurs over the biliary ducts (having a color outside the target color range), but is attenuated over the liver, which has a color targeted for attenuation of the fluorescence pseudo-color overlay.

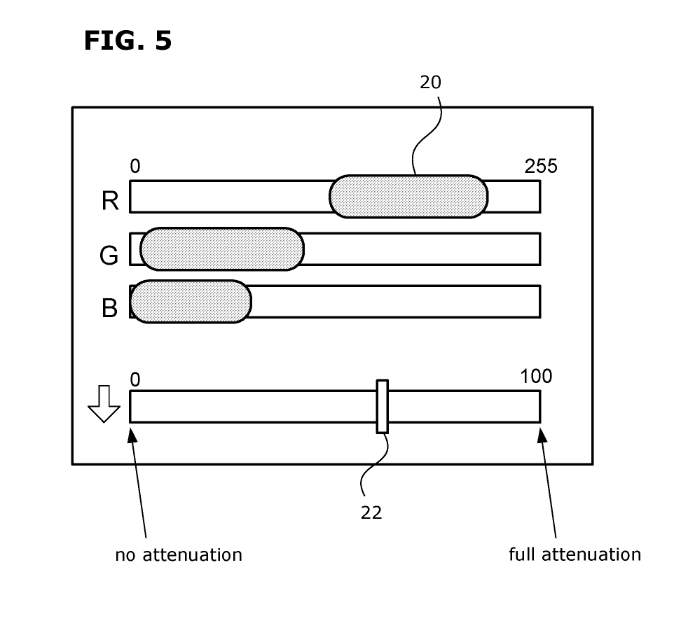

2(b). Manner of Selecting. The target color range can be selected by the user. This may be performed in any suitable way, such as using a color range selection tool. For example, as explained above, FIGS. 4A and 4B show how a user can select a target color range by marking portions of the color image itself. Another type of color selection tool that could be used is a graphical user interface that provides a mechanism to select colors (e.g. by using a color palette, using a color picker or ink dropper, using sliding bars, etc.). FIG. 5 shows an example of a color range selection tool (as a graphical user interface) for adjusting the target color range. This color tool defines colors according to the RGB scale (Red-Green-Blue). Each of the RGB values are adjusted on a horizontal scale (0-255) with a slider bar 20. Each of the slider bars 20 can be shifted left/right and widened/narrowed to select the target color range.

In the example of FIG. 5, the selected color range represents colors over which the fluorescence image overlay should be attenuated. At the bottom of the color selection tool, there is also a slider bar 22 for adjusting the amount of attenuation desired, with zero being fully opaque (no attenuation) and 100 being complete transparency (full attenuation) of the pseudo-color overlay. The selected color range can be defined according to any other color coordinate system as well, such as HSL color space (Hue-Saturation-Lightness) or HSV color space (Hue-Saturation-Value).

2(c). Preset Selection. In some embodiments, the target color range is preselected. The preselected target color range can be for the color of certain body tissue (e.g. fatty tissue, serosal tissue, liver tissue, etc.). For example, the preselected target color range may be the range of colors for human intra-abdominal fatty tissue, which generally has a yellow-reddish hue, with moderate to high saturation and moderate to high luminance. In some cases, in HSL coordinates, the preselected target color range are the colors having a hue value in the range of 20-80, saturation of 40% or greater, and lightness of 30% or greater. The preselected target color range may be any set of colors in this range.

For example, the preselected target color range may be (in HSL) hue 30-60, saturation 60-90%, and lightness 50-90%.

In another example, the preselected target color range may be the range of colors for human liver tissue, which generally has an orange to violet hue with saturation in the low to medium range and lightness in the low to medium range. In some cases, in HSL coordinates, the preselected target color range are the colors having a hue value in the range of 280-[36010]-40, saturation of 80% or less, and lightness of 70% or less. The preselected target color range may be any set of colors in this range. For example, the preselected target color range may be (in HSL) hue 300-20, saturation 20-60%, and lightness 20-60%.

A person skilled in the art would understand that different color coordinate systems are inter-convertible, i.e. coordinates in one color space system can be translated to its equivalent coordinate in a different color space system. For the above-specified HSL color coordinates, my invention encompasses the equivalent coordinates in different color coordinate systems. The user may further be allowed to adjust the preset color selections as desired.

3. Selective Fluorescence Imaging. The photophysical characteristics of the NIR fluorescent dye can be affected by its chemical environment. See Baohong Yuan et al, “Emission and absorption properties of indocyanine green in Intralipid solution” in Journal of Biomedical Optics, vol 9(3):497-503 (May/June 2004); Jarmo Alander et al, “A Review of Indocyanine Green Fluorescent Imaging in Surgery” in International Journal of Biomedical Imaging, Article ID 940585 (2012); John Kraft et al, “Interactions of Indocyanine Green and Lipid in Enhancing Near-Infrared Fluorescence Properties: The Basis for Near-Infrared Imaging in Vivo” in Biochemistry, vol 53:1275-1283 (2014); Mikhail Berezin et al, “Near-Infrared Fluorescence Lifetime pH-Sensitive Probes” in Biophysical Journal, vol 100(8):2063-2072 (April 2011); Chintan Trivedi, “Development of a Time Resolved Fluorescence Spectroscopy System for Near Real-Time Clinical Diagnostic Applications,” Thesis for Master's Degree at the Texas A&M University (May 2009); Mikhail Berezin et al, “Near Infrared Dyes as Lifetime Solvatochromic Probes for Micropolarity Measurements of Biological Systems” in Biophysical Journal, vol 93(8):2892-2899 (October 2007); the disclosures of these literature references are incorporated by reference herein.

One example of a photophysical characteristic that could be affected by the chemical environment is shifting of the excitation or emission spectra of the fluorescent dye. Another example of a photophysical characteristic that could be affected is a change in the fluorescence lifetime of the fluorescent dye (e.g. resulting from changes in the rate of decay).

Environmentally-induced changes in the photophysical characteristics of the fluorescent dye may allow for selective imaging of the fluorescence emission at a particular region of interest in the patient's anatomy. For example, the pH of liver bile is different from the pH in the biliary ducts. Also, the biochemical composition of liver bile is different than in the biliary ducts, such as in the types and amounts of bile salts, bilirubin, types of fats (cholesterol, fatty acids, and lecithin), and inorganic salts. Also, the concentration of the fluorescent dye in liver bile may be different than its concentration in the biliary ducts. As a result, the photophysical characteristics of the NIR fluorescent dye present in the liver may be different from the photophysical characteristics of the same fluorescent dye present in the biliary ducts. These differing photophysical characteristics can allow for selective imaging of the biliary ducts.

As used herein, the term “fluorescence imaging condition” means a condition based on the photophysical characteristics of the fluorescent dye under which the fluorescence imaging takes place that the user can select to control the fluorescence imaging (e.g. to enhance or attenuate the fluorescence image). The fluorescence imaging condition is selected from among multiple possible selections, which may be continuous, discrete, etc. Examples of fluorescence imaging conditions include the wavelength for the excitation light, the wavelength for detection of the fluorescence emission light, and the time or modulation frequency for detecting the fluorescence emission light (in fluorescence lifetime imaging).

Any suitable type of user controller can be provided on the apparatus. For example, the user controller may a mechanical controller (such as a dial connected to a filter wheel), electromechanical controller (such as slide that electrically controls a monochromator), digital controller (such as a touchscreen control that adjusts a time delay selection in fluorescence lifetime imaging), or combinations thereof.

As used herein, the term “detecting/detection” in the context of selective imaging by the selection of fluorescence imaging conditions does not necessarily mean that fluorescence signals outside of the selected conditions are not read, acquired, etc. Such out-of-range signals may still be read or acquired, but are not considered or are disregarded in the process of selective fluorescence imaging. For example, in the case of fluorescence lifetime imaging, fluorescence signal readings may be taken continuously, but only signals after a selected time point may be processed to generate the image. Selective imaging of a part of the surgical field does not necessarily mean that the non-selected part is completely eliminated. For example, it is possible that the fluorescence image at the non-selected part is less intense, faded, or less uniform (e.g. patchy).

The user's selection of the fluorescence imaging condition(s) may result in the fluorescence image at a first part of the surgical field being enhanced or attenuated relative to the fluorescence image at a second part of the surgical field. For example, selective imaging of an area of interest in the surgical field may be achieved by the user selecting fluorescence imaging condition(s) to increase the intensity of the fluorescence pseudo-color at an area of interest relative to a part that is outside the area of interest. In another example, selective imaging of an area of interest in the surgical field may be achieved by the user selecting fluorescence imaging condition(s) to attenuate the intensity of the fluorescence pseudo-color at a part that is outside the area of interest to selectively image the area of interest. A combination of both enhancing and attenuating may also be performed.

In some embodiments, the first part of the surgical field comprises at least a portion of the common bile duct, but not the liver; and the second part of the surgical field comprises at least a portion of the liver, but not the common bile duct. In some cases, the fluorescence image at the first part is enhanced relative to the fluorescence image at the second part. In some cases, the fluorescence image at the second part is attenuated relative to the fluorescence image at the first part. A combination of both enhancing and attenuating may also be performed.

A selective fluorescent video image of the surgical field is composited with a color video image of the surgical field in real-time to produce a composited video image. The composite video image combines a visible color image and a selective fluorescence image of the surgical field. In the composited video image, the selective fluorescence video image (which is in greyscale) is shown in a pseudo-color.

3(a). Spectrally-resolved fluorescence imaging. Selective imaging may be performed by spectrally-resolved fluorescence imaging. In this type of selective imaging, the differing spectral characteristics of the fluorescent dye at one part of the surgical field relative to another part of the surgical field is distinguished by spectrally-resolved fluorescence imaging (such as differences in the excitation spectrum or the emission spectrum).

As used herein, the term “spectrally-resolved fluorescence imaging” means the technique of providing different fluorescence images based on the different spectral characteristics of the dye, such as the fluorescence excitation spectrum or the fluorescence emission spectrum. With spectrally-resolved fluorescence imaging, selective imaging may be achieved by selecting a wavelength for the excitation light or a wavelength for detection of the fluorescence emission light. The wavelength is selected from among multiple possible wavelengths for the fluorescence excitation light or the fluorescence emission detection. As used herein, the term “select/selecting a wavelength” encompasses selecting a single wavelength as well as selecting multiple wavelengths or a range of wavelengths, such as selecting wavelengths longer than a minimum cut-off (longpass), selecting wavelengths shorter than a maximum cut-off (shortpass), or selecting a band of wavelengths (bandpass).

Different excitation light wavelengths can be made selectable in any suitable way. For example, different excitation light sources may be used (e.g. an array of light-emitting diodes that produce light at different wavelengths). In another example, a single excitation light sources coupled to an adjustable wavelength selector could be used. In another example, combinations of such techniques could be used.

Detection of different wavelengths for fluorescence emission can be performed in any suitable way. For example, there may be an array of light sensors (e.g. CCD cameras) coupled to different wavelength selectors (e.g. different optical filters). In this parallel arrangement, different wavelengths can be selected by selecting the light sensor for image acquisition. In another example, a single light sensor coupled to an adjustable wavelength selector could be used. In another example, combinations of such techniques could be used.

Selection of the wavelength can be performed by any suitable technique, including digital processing techniques (such as Fourier transform techniques), optical techniques (including optoelectronic), or combinations thereof. Using an adjustable wavelength selector is a common way to select for desired wavelengths. Examples of adjustable wavelength selectors include optical filters, prisms, monochromators, polychromators, diffraction gratings, acoustic-optic tunable filters, liquid crystal tunable filters, angle-tuned thin film filters, etc. Such wavelength selectors are available in various types of spectral measurement instruments, such as spectrographs, spectrometers, spectrophotometers, or spectral analyzers.

The adjustable wavelength selector may comprise one or more different components. For example, the adjustable wavelength selector may be a single unit (such as a single optical filter that provides multiple bandpasses) or an assembly of components that function together (such as a set of different bandpass filters mounted on a filter wheel). The wavelengths provided by the adjustable wavelength selector may be continuous, discrete, etc.

In some embodiments, an adjustable wavelength selector is used to select a wavelength (e.g. providing a selection from among multiple wavelengths or multiple wavelength bands). The adjustable wavelength selector may be applied to the excitation light or the detection of emission light. In some cases, the adjustable wavelength selector provides a selection from among at least three wavelength bands that are continuously adjacent or overlapping with each other; in some cases, at least four; and in some cases, at least five wavelength bands that are adjacent or overlapping with each other. Having a greater number of wavelength bands for selection can be useful for tuning the excitation light or the emission detection to distinguish small changes in the photophysical characteristics of the NIR fluorescent dye.

In some cases, each of the wavelength bands has a width of less than 25 nm. Having narrower bands can be useful for tuning the excitation light or the emission detection to distinguish small changes in the photophysical characteristics of the near-infrared fluorescent dye.

In some embodiments, the adjustable wavelength selector provides a selection that comprises multiple possible wavelength bands (e.g. by providing multiple bandpass filters). The multiple possible wavelength bands are within the range of 700-900 nm. The selection may further include other wavelength bands outside of this range.

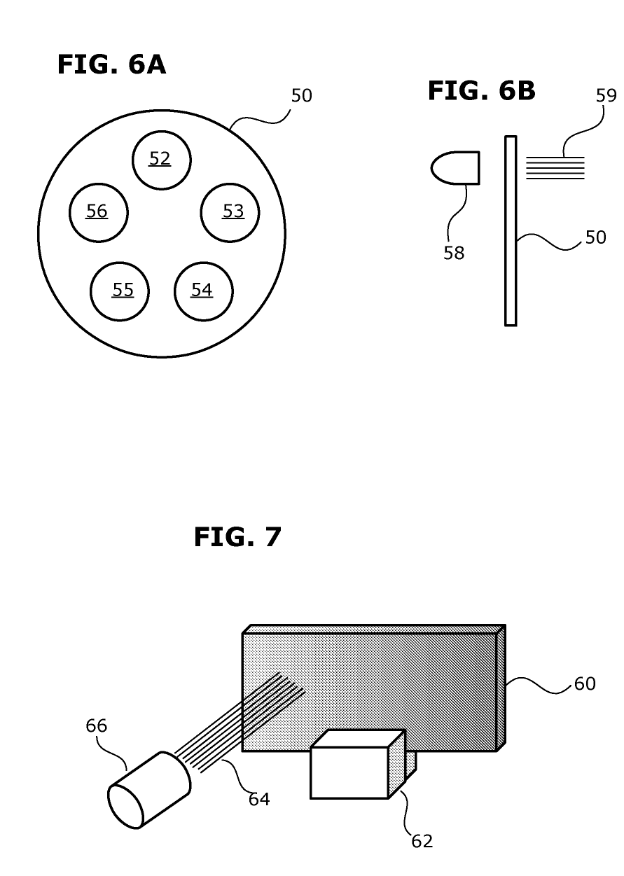

For example, FIGS. 6A and 6B show a filter wheel 50 that provides multiple optical filters. As shown in FIG. 6A, a set of optical filters 52-56 are mounted on the filter wheel 50, which is electronically controlled. The set of filters span a wavelength range that is within the range of 700-900 nm. Filter 52 is a 790-805 nm bandpass filter; filter 53 is a 805-817 nm bandpass filter; filter 54 is a 815-825 nm bandpass filter; filter 55 is a 823-835 nm bandpass filter; and filter 56 is a 835-850 nm bandpass filter.

Here, the 790-805 nm band of filter 52 is adjacent the 805-817 nm band of filter 53, which is overlapping with the 815-825 nm band of filter 54, which is overlapping with the 823-835 nm band of filter 55, which is adjacent with the 835-850 nm band of filter 56. As shown in FIG. 6B, a fluorescence light sensor 58 is placed behind the filter wheel 50. The wavelength of fluorescence emission light 59 from the surgical field being transmitted to light sensor 58 can be selected by turning the filter wheel 50.

FIG. 7 shows an example of a linear filter 60 that gradually changes the wavelength from 740 nm at one end to 800 nm at the other. The linear filter 60 is mounted on a motorized platform 62 that can shift the filter 60 back and forth. Excitation light source 66 is filtered by linear filter 60. The wavelength excitation light 64 can be selected by shifting the linear filter 60 back and forth.

Sung Chang et al, “Optimal Excitation Wavelengths for Discrimination of Cervical Neoplasia” in IEEE Transactions on Biomedical Engineering, vol 49(10):1102-1110 (October 2002) is incorporated by reference herein. Chang describes a fluorescence spectroscopic system that provided excitation light at 16 different wavelengths. The fluorescence emission was measured at 50 to 130 points within a range of 380 to 910 nm in 5 nm increments. A Xenon arc lamp coupled to a monochromator provided the excitation light. Fluorescence intensity was recorded by a charge-coupled device camera as a function of emission wavelength selected by a polychromator.

Diana de Veld et al, “Clinical study for classification of benign, dysplastic, and malignant oral lesions using autofluorescence spectroscopy” in Journal of Biomedical Optics, vol 9(5):940-950 (September/October 2004) is incorporated by reference herein. Veld describes measurements of fluorescence at different wavelengths as produced by different excitation wavelengths. The wavelength for the excitation light was selected by a monochromator. The fluorescence emission was measured by spectrograph instrument.

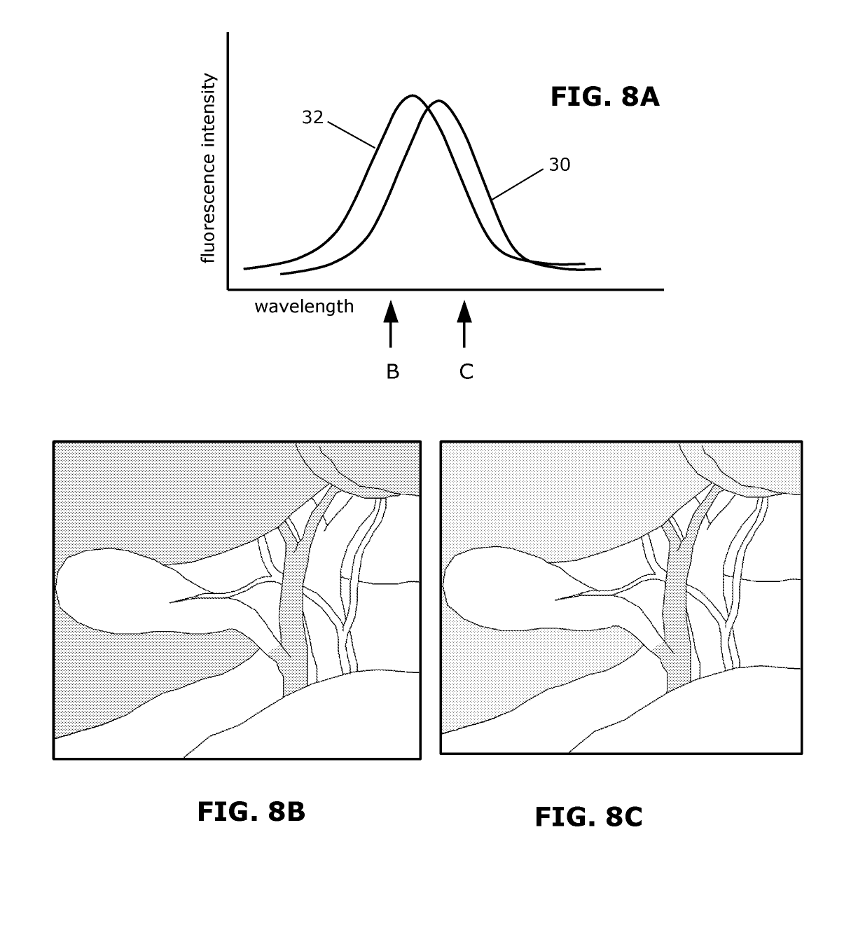

FIGS. 8A-C show an example of how selecting the wavelength for emission detection can allow selective imaging of the biliary ducts. FIG. 8A shows a plot of the fluorescence intensity (y-axis) at different detection wavelengths (x-axis) for a fluorescent dye. Line 32 is the emission spectrum from the dye that is present in the liver. Line 30 is the emission spectrum from the dye that is present in the biliary ducts. As seen here for this example, the emission spectrum from the dye, for whatever reason, is different in the biliary ducts than in the liver. With wavelength B, fluorescence emission from both the liver and the biliary ducts are captured. FIG. 8B shows the resulting fluorescence image of the surgical field in green pseudo-color. When the emission detection is adjusted to wavelength C, less of the fluorescence emission from the liver is captured compared to the biliary ducts. FIG. 8C shows the resulting fluorescence image of the surgical field, in which the fluorescence image of the liver is attenuated to allow selectively viewing of the biliary ducts.

3(b). Time-resolved fluorescence imaging. Selective imaging may be performed by time-resolved fluorescence imaging. Differences in fluorescence lifetime can be resolved in the time domain or in the frequency domain. In this type of selective imaging, the differing fluorescence lifetime characteristics of the fluorescent dye at one part of the surgical field relative to another part of the surgical field is distinguished by time-resolved fluorescence imaging.

As used herein, the term “time-resolved fluorescence imaging” means the technique of providing different fluorescence images based on the different fluorescence lifetimes of the dye. The dye may have a shorter lifetime or a longer lifetime in different circumstances. With time-resolved fluorescence imaging, selective imaging may be performed by selecting the time (for the time domain approach) or the frequency for pulsing or modulating the excitation light (for the frequency domain approach). As used herein, the term “select/selecting a time” in the context of fluorescence lifetime imaging encompasses selecting a single time point as well as a range of times, such as a period after a time point, a period before a time point, or a period within a window of time. As used herein, the term “select/selecting a frequency” in the context of fluorescence lifetime imaging encompasses selecting a particular frequency as well as selecting ranges of frequencies, such as frequencies higher than a minimum, frequencies lower than a maximum, or a band of frequencies.

In some embodiments, the fluorescence imaging is performed by selecting a time (from among multiple possible times) for detecting the fluorescence emission after a pulse of excitation light. In some embodiments, the fluorescence imaging is performed by selecting a frequency for pulsing or modulating the excitation light.

Sylvain Gioux et al, “Low-frequency wide-field fluorescence lifetime imaging using a high-power near-infrared light-emitting diode light source” in Journal of Biomedical Optics, vol 15(2):026005 (March/April 2010) is incorporated by reference herein. Gioux implemented a frequency-domain approach to fluorescence lifetime imaging. Excitation light was provided by a near-infrared LED light source, or with a laser diode for comparison. The intensity of the excitation light was modulated at a frequency of 35 MHz. The apparatus was able to detect a 0.5 ns difference in fluorescence lifetime.

Hugh Sparks et al, “A flexible wide-field FLIM endoscope utilising blue excitation light for label-free contrast of tissue” in J. Biophotonics, vol. 8(1-2):168-178 (2015) is incorporated by reference herein. Sparks describes a real-time fluorescence lifetime imaging endoscope that implemented a time-gated (time domain) approach. The apparatus used a programmable rapidly-switchable delay generator to control the delay between the excitation pulse and the gating of a gated optical intensifier that was coupled to CCD camera. The gated optical intensifier was is triggered to acquire a series of time-gated fluorescence intensity images at increasing delays after the excitation pulses.

Chintan Trivedi, “Development of a Time Resolved Fluorescence Spectroscopy System for Near Real-Time Clinical Diagnostic Applications,” thesis for Master of Science at Texas A&M University (May 2009) is incorporated by reference herein. Trivedi reports a time-resolved fluorescence spectroscopy system that had a dual grating spectrograph with monochromator for selecting the excitation light wavelength, a high speed digital oscilloscope for sampling and recording data, and a delay generator for synchronizing the detection. The digital delay generator sends pulses to trigger the excitation light source, and after a selected delay time, send pulses to gate the light sensor for detection.

Shuna Cheng et al, “Flexible endoscope for continuous in vivo multispectral fluorescence lifetime imaging” in Optics Letters, vol 38(9):1515-1517 (May 2013). Cheng describes a fluorescence lifetime imaging endoscope that used an intensified CCD camera and time-gated imaging after 10 kHz laser pulses.

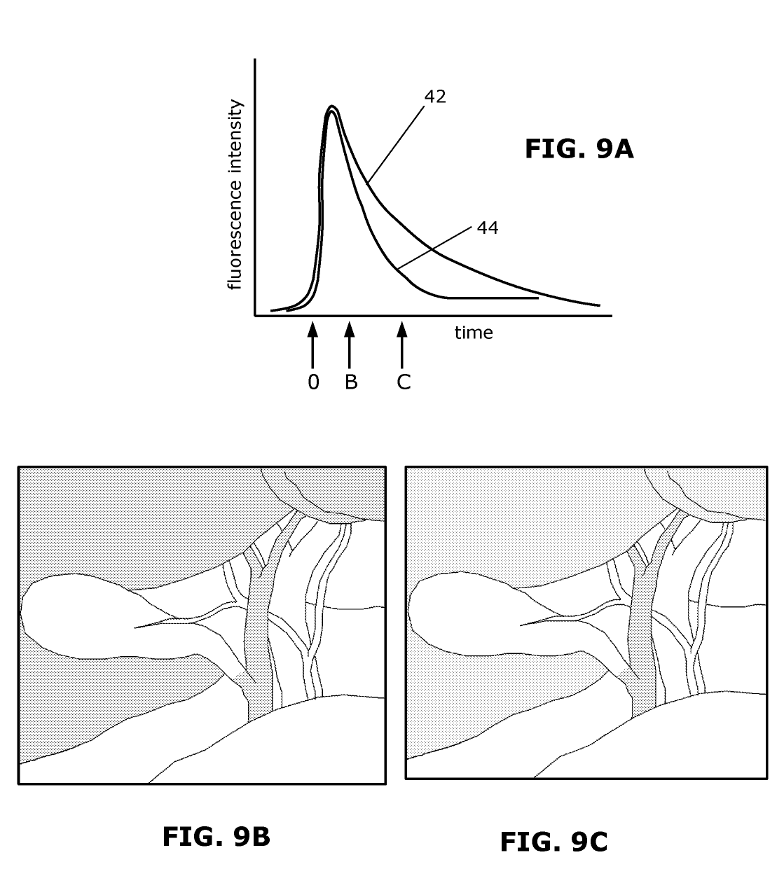

FIGS. 9A-C show an example of how selecting the time for detecting fluorescence emission can allow for can allow selective imaging of the biliary ducts. FIG. 9A shows a plot of the fluorescence intensity (y-axis) at different detection times (x-axis) after photoexcitation of a fluorescent dye. The dye is exposed to a pulse of excitation light at time=0. Line 42 is the fluorescence signal from the dye present in the biliary ducts; line 44 is the fluorescence signal from the dye present in the liver. As seen here for this example, for whatever reason, the fluorescence signal from the liver decays faster than the fluorescence signal from the biliary ducts. At time point B, the fluorescence signal from both the liver and the biliary ducts are captured. FIG. 9B shows the resulting fluorescence image of the surgical field in green pseudo-color. When the time delay is adjusted to time point C, less of the fluorescence emission from the liver is captured compared to the biliary ducts. FIG. 9C shows the resulting fluorescence image of the surgical field, in which the fluorescence image of the liver is attenuated to selectively view the fluorescence image of the biliary ducts.

4. Imaging System. The visible light and NIR fluorescence are captured by an imaging system and processed for display on a display screen. The imaging system comprises one or more digital image sensors for capturing the images. The imaging system may have multiple image sensors (e.g. one for visible color and another for NIR light). However, having multiple image sensors is not necessary. For example, a single multi-modal camera that captures both visible light and NIR light may be used. Any suitable type of digital image sensor may be used including the commonly-used charge-coupled devices (CCD) or CMOS sensors (complementary metal-oxide semiconductor).

The image sensor may be a component of a digital video camera. Any suitable color digital video camera equipment may be used for capturing the color images from the surgical field. Any suitable NIR/IR-light digital video camera equipment may be used for capturing the NIR light spectrum. One example of a NIR/IR video camera that may be suitable for use is Hamamatsu Photonics' Orca-R2 near-infrared camera.

The image sensor(s) may be situated at any suitable location relative to the endoscope. For example, in a conventional fiber optic endoscope, a video camera may be coupled to the endoscope at its proximal end. In another example, for a digital endoscope, an image sensor may be attached at the distal end of the endoscope.

The signals from the image sensor(s) are processed into video images. The color video images and the NIR fluorescence video images are composited together to generate a composite video image. The imaging system also comprises the necessary hardware/software to process the captured images into moving (video) images and display the video on a display screen. The imaging system may also provide other additional functions such as printing images onto paper medium or storing the video images on a mass storage device (such as CD, DVD, solid-state drive, or hard drive). The imaging system may provide various other conventional image processing capabilities, including color adjustment, brightness adjustment, contrast adjustment, frame rate adjustments, digital filtering, gain adjustment, or color balancing. In addition to displaying the composited video image, other types of images may also be displayed on the display screen, such as a separate frame for color images, a separate frame for NIR fluorescence images, or a separate frame for side-by-side images.

5. Optical Hardware. One or more light sources are used to illuminate the surgical field and excite the fluorescent dye. Broad spectrum white light is used to illuminate the surgical field to produce color video images (also known as visible light imaging or RGB imaging). The surgical field is also irradiated with fluorescence excitation light to excite the fluorescent dye and cause it to fluoresce NIR light. Any suitable type of white light or NIR light source may be used, such as a halogen lamp, arc lamp (e.g. xenon), laser diode, or light-emitting diode (LED).

The white light and the fluorescence excitation light may be provided from a single source (i.e. the same light source provides both white light and the fluorescence excitation light) or from different sources. Other optical hardware may be used to perform various functions such as connecting the light source(s), connecting the image sensor(s), adjusting the wavelength range of the illumination or excitation light, extracting the NIR fluorescence light, direct outgoing or incoming light in different directions, etc. Examples of optical hardware that may be used include light mixers, light couplers, filters (long-wavelength pass, short-wavelength pass, bandpass), dichroic mirrors, prisms, lenses, beam splitters, collimators, etc.

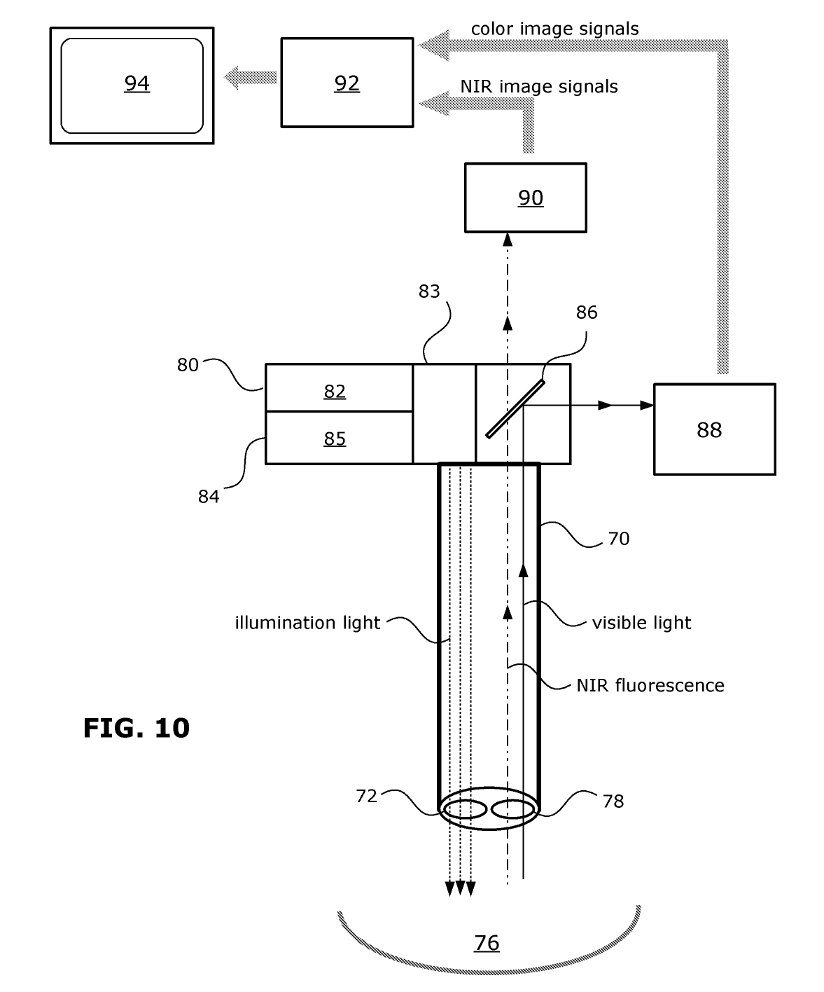

6. Example Apparatus & System. FIG. 10 shows an example apparatus of my invention. In this example, fluorescent indocyanine green (ICG) dye has been administered intravenously to the patient. This fluorescent dye has its peak spectral absorption at about 800 nm and produces fluorescence emission with a peak wavelength of about 810 to 830 nm. The fluorescent dye is present in the surgical field which is viewed through the laparoscope tube that is inserted into the patient's abdomen.

The laparoscope tube 70 has a hollow lumen (illumination channel 72) that contains a fiber optic bundle for conducting illumination light to illuminate the surgical field 76 as well as fluorescence excitation light for exciting the fluorescent dye. The laparoscope tube 70 also has an imaging channel 78 to receive reflected visible light from the surgical field 76 as well as the NIR light emitted from the fluorescent dye.

The white light illumination source 80 is a halogen lamp 82 that generates broad spectrum white light. The halogen lamp 82 is coupled to a short-pass (wavelength) filter in optical coupler 83 that filters out NIR light with wavelengths greater than 700 nm. This depletion of NIR-range wavelengths reduces interference with the fluorescence emission image coming from the surgical field. The NIR excitation light source 84 is a laser diode 85 that emits 770 nm NIR light. Both this visible light and the NIR excitation light are transmitted by a fiber optic bundle through the illumination channel 72 of the laparoscope 70 onto the surgical field 76. The excitation light causes the ICG dye to fluoresce with NIR light.

Light from the surgical field 76 (both reflected visible light and NIR fluorescence) are transmitted through the imaging channel 78 of the endoscope. A dichroic mirror 86 (acting as a beam splitter) reflects light having wavelengths shorter than 700 nm (visible light) towards the color video camera apparatus 88 and transmits light having wavelengths longer than 700 nm towards the NIR light camera apparatus 90. The color video camera 88 is fitted to a 400-650 nm bandpass filter to capture the visible light spectrum. The NIR light camera 90 is fitted to a 790-830 nm bandpass filter to capture the NIR light fluoresced by the dye.

Image data from the color video camera 88 and NIR camera 90 are received by the image processing component 92, which performs the image processing that combines the images from the two channels for superimposed display on the display screen 94. In some embodiments, the display screen 94 is a touchscreen display screen. This may facilitate user interactions with the fluorescence overlay window portion or attenuation of the fluorescence overlay, such as selecting target color ranges or moving the window portion for the NIR fluorescence overlay.

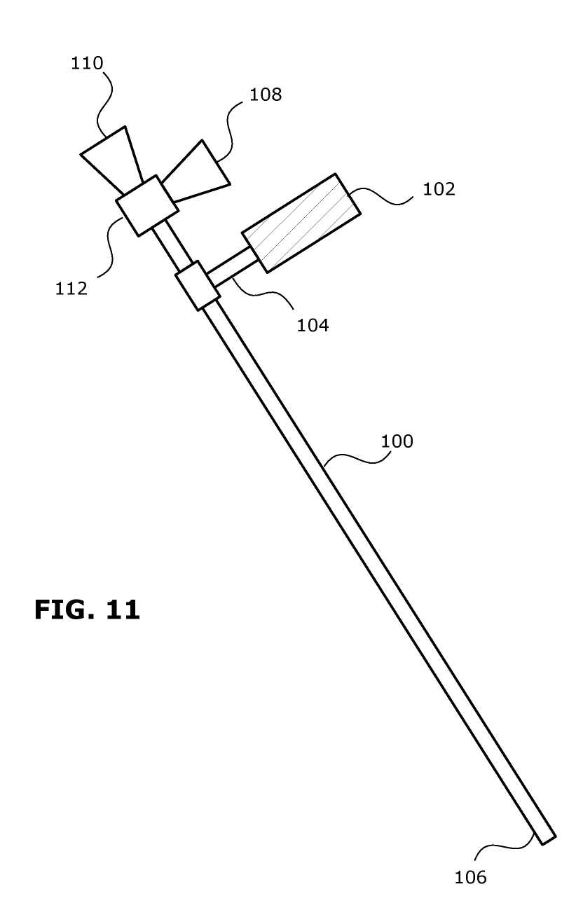

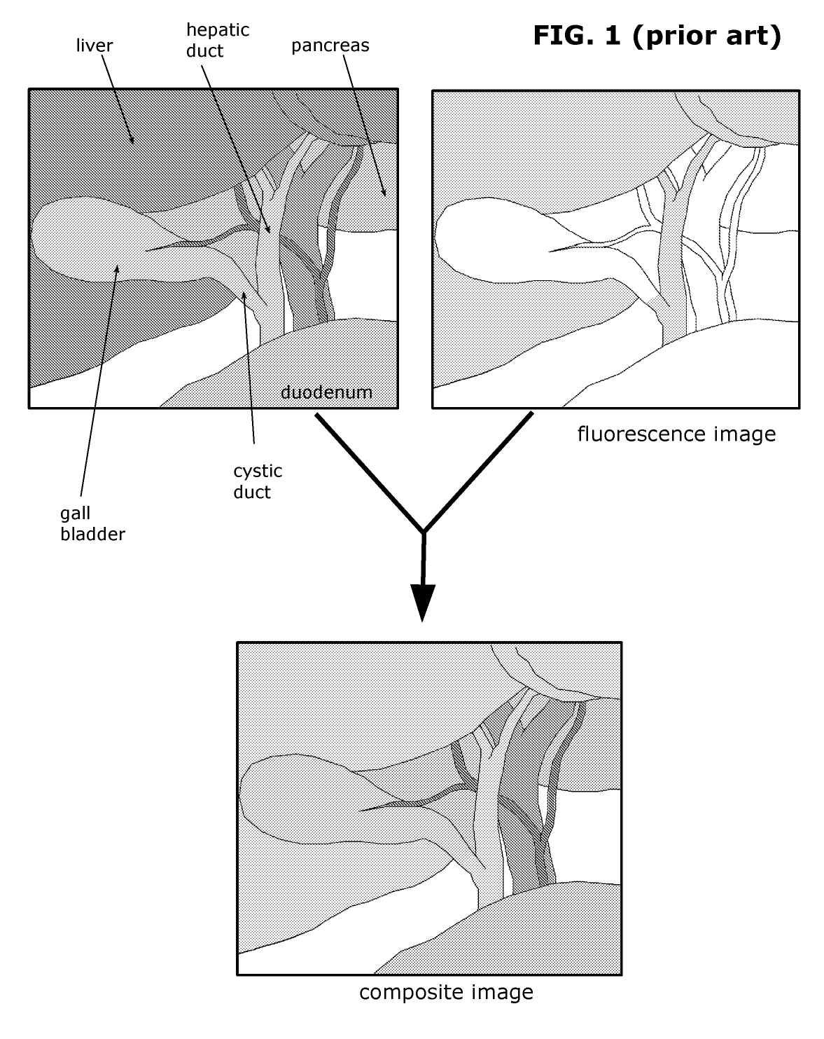

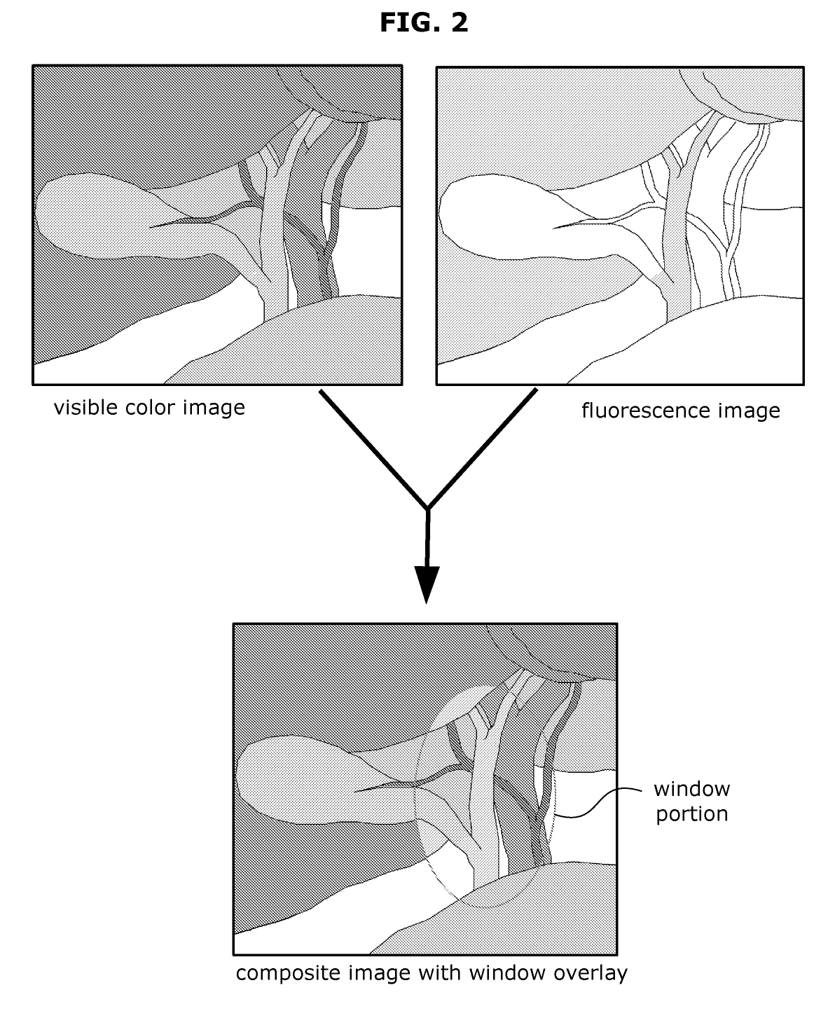

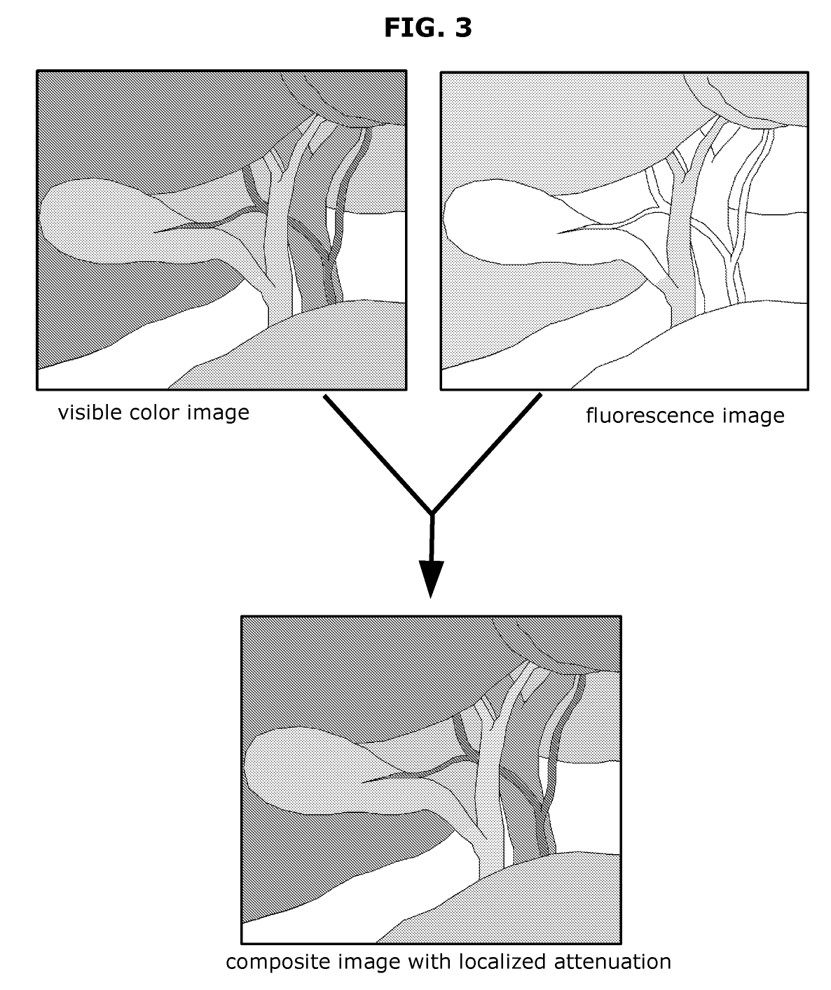

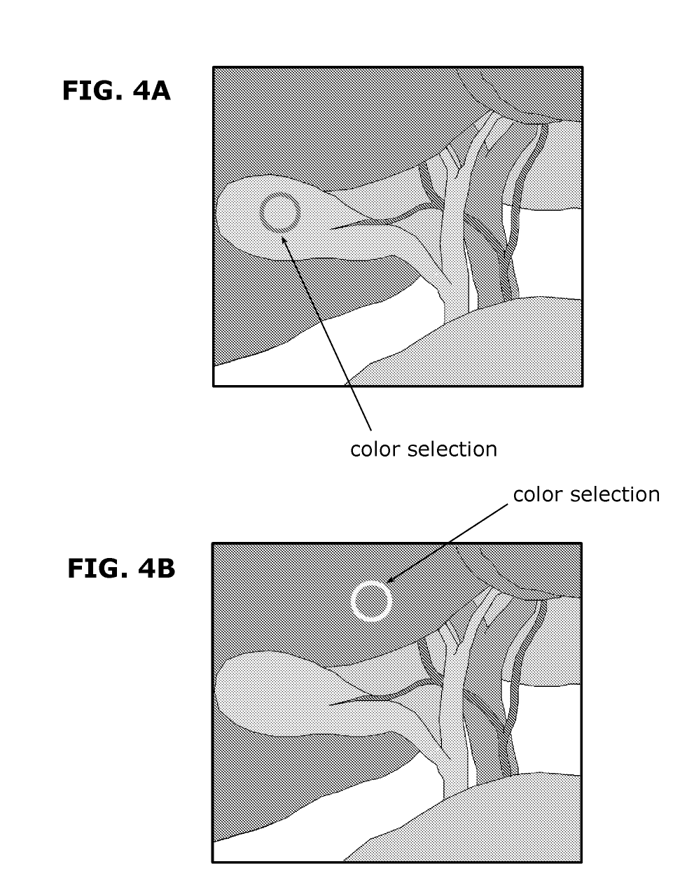

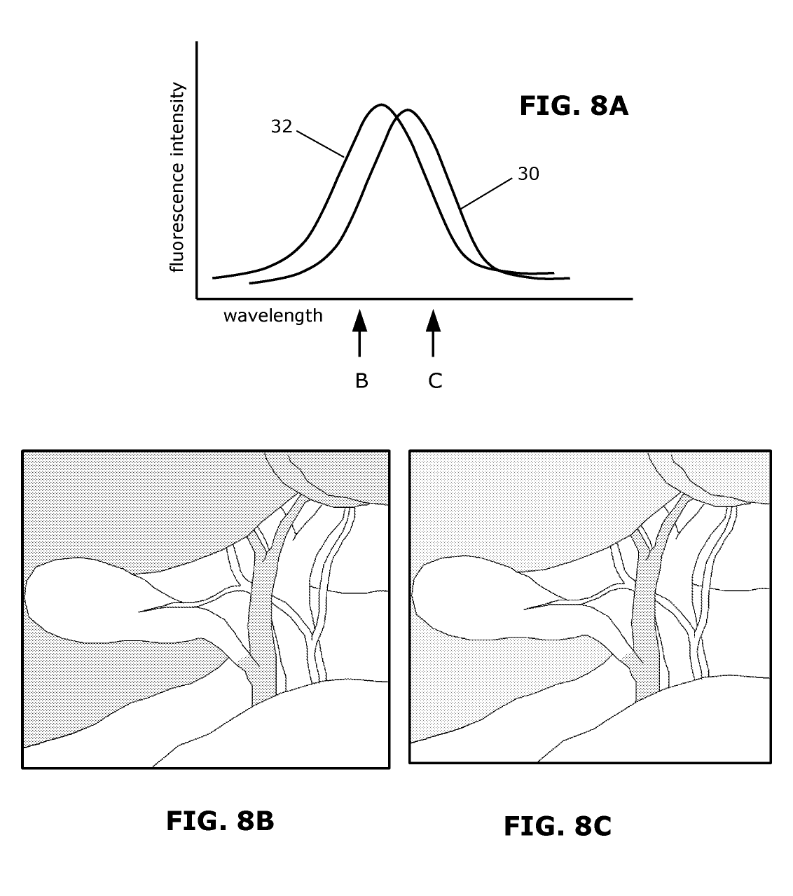

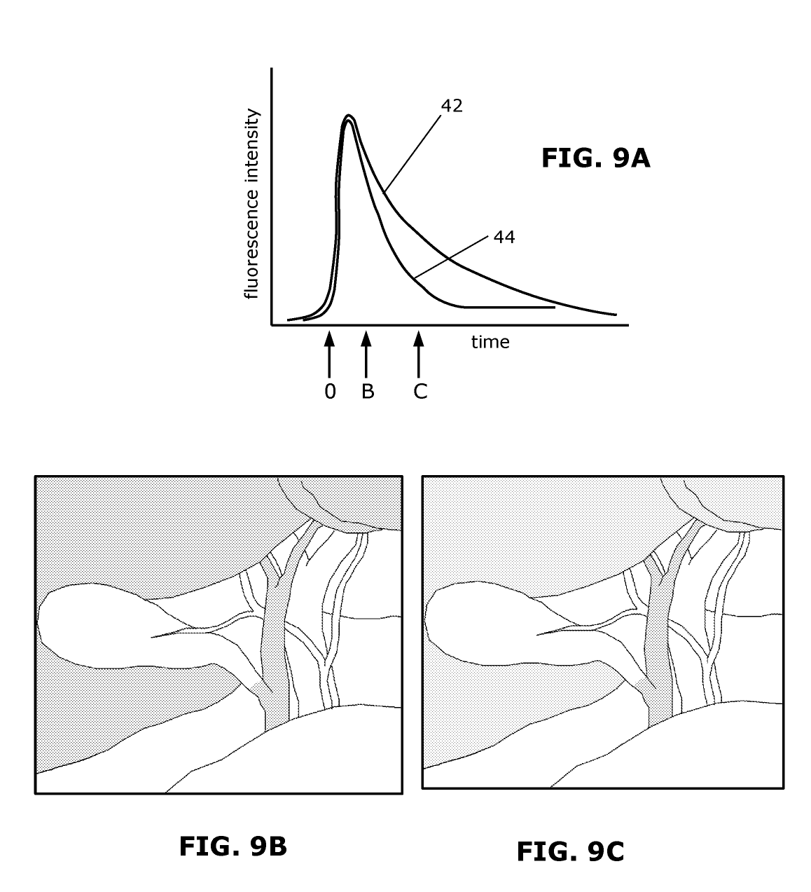

FIG. 11 shows an example laparoscope set-up of my invention. A combined illumination/excitation light source 102 is coupled to a laparoscope tube 100 (i.e. a rigid endoscope for use in the abdomen or pelvis) via a light source adapter 104. The light generated by the light source 102 is emitted out of the distal end 106 of the laparoscope tube 100. An adapter 108 for a color video camera and an adapter 110 for an NIR camera are coupled to a beam splitter 112 (e.g. right-angle prism, half-silvered mirror, dichroic mirrored prism, etc.). Light from the surgical field travels through the laparoscope tube 100 to the beam splitter 112. At the beam splitter 112, the visible light spectrum is reflected towards the color video camera coupled to the adapter 108 while the NIR light is transmitted to the NIR camera coupled to the NIR camera adapter 110.

7. Surgical Method. Another aspect of my invention is a surgical method. In the surgical method, a fluorescent dye is orally or intravenously administered to the patient. Of the many different NIR fluorescent dyes, indocyanine green (ICG) and methylene blue are the only two that are FDA-approved for clinical use today. But other NIR fluorescent dyes that can be used include those based on derivatives of cyanine, phthalocyanines, porphyrin, squaraine, borondipyrromethane (BODIPY), benzo[c]heterocycles, and xanthenes, such as those described in Escobedo et al., “NIR Dyes for Bioimaging Applications” in Chemistry Faculty Publications & Presentations, Paper 56 (2010). In some embodiments, those NIR fluorescent dyes that are primarily excreted through the biliary system are preferred for use in my invention.

An endoscope is introduced into the patient's body (e.g. inside the abdomen) to view a surgical field inside the patient's body. The endoscope may be a laparoscope (rigid endoscope) or any other type of endoscope. The surgical field is exposed to white light and fluorescence excitation light. In some embodiments, the white light or the fluorescence excitation light is transmitted through the endoscope. In some embodiments, the surgical procedure is a cholecystectomy and the surgical field includes the biliary ducts. Via the endoscope, a color image and NIR fluorescence image of the surgical field are received. The color images and the fluorescence images are composited and processed into a composite video image.

The clinician performs the surgery using the composite video image for guidance. For example, in a cholecystectomy procedure, the clinician may view one or more of the biliary ducts that is shown by fluorescence image overlay in the composite video image to assist with dissection of the gallbladder or bile duct.

The foregoing description and examples have been set forth merely to illustrate my invention and are not intended to be limiting. Each of the disclosed aspects and embodiments of my invention may be considered individually or in combination with other aspects, embodiments, and variations of my invention. In addition, unless otherwise specified, the steps of the methods of my invention are not confined to any particular order of performance. Modifications of the disclosed embodiments incorporating the spirit and substance of my invention may occur to persons skilled in the art, and such modifications are within the scope of my invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof. However, in another example, the expression “either A or B” means only A or B, but not both.

1. A method of performing an endoscopic surgery on a patient, comprising:
administering a near-infrared fluorescent dye to the patient;
inserting an endoscope into the patient's body to view a surgical field;
exposing the surgical field with white light and fluorescence excitation light;
viewing on a display screen, a composite video image that combines a visible color image and a fluorescence image of the surgical field viewed from the endoscope, wherein (a) the fluorescence image is limited to only a window portion of the composite video image that is less than the full size of the composite video image; or (b) the fluorescence image is attenuated over a portion of the color image on the basis of the color of that portion of the color image; and
performing the endoscopic surgery using the composite video image for guidance.

2. The method of claim 1, wherein the fluorescence image is limited to only a window portion of the composite video image that is less than the full size of the composite video image

3. The method of claim 2, further comprising selecting the size of the window portion.

4. The method of claim 2, further comprising selecting the position of the window portion.

5. The method of claim 2, further comprising selecting the shape of the window portion.

6. The method of claim 2, wherein the surgical procedure is a cholecystectomy and the surgical field includes one or more of the patient's biliary ducts.

7. The method of claim 6, wherein the step of performing the endoscopic surgery comprises viewing one or more of the biliary ducts that is shown by the fluorescence image in the composite video image to assist with dissection of the patient's gallbladder or biliary duct.

8. The method of claim 6, further comprising selecting the size of the window portion, selecting the position of the window portion, or selecting the shape of the window portion to encompass one or more of the biliary ducts.

9. The method of claim 2, wherein the window portion is not more than 75% of the full size of the composite video image.

10. The method of claim 2, wherein the window portion is not more than 60% of the full size of the composite video image

11. The method of claim 1, wherein the fluorescence image is attenuated over a portion of the color image on the basis of the color of that portion of the color image.

12. The method of claim 11, further comprising selecting a target color range, and wherein the fluorescence image is attenuated over portions of the color image having a color in the target color range.

13. The method of claim 11, further comprising selecting a target color range, and wherein the fluorescence image is attenuated over portions of the color image not having a color in the target color range.

14. The method of claim 11, wherein the surgical procedure is a cholecystectomy and the surgical field includes one or more of the patient's biliary ducts.

15. The method of claim 14, wherein the step of performing the endoscopic surgery comprises viewing one or more of the biliary ducts that is shown by the fluorescence image in the composite video image to assist with dissection of the patient's gallbladder or biliary duct.

16. The method of claim 11, wherein the fluorescence image is attenuated over portions of the color image either (i) having a color in a target color range or (ii) not having a color in the target color range.

17. The method of claim 16, wherein the target color range is predetermined.

18. The method of claim 11, further wherein the fluorescence image is limited to only a window portion of the composite video image that is less than the full size of the composite video image.

19. The method of claim 18, further comprising selecting the size of the window portion, selecting the position of the window portion, or selecting the shape of the window portion.

20. The method of claim 19, wherein the fluorescence image is attenuated over portions of the color image either (i) having a color in a target color range or (ii) not having a color in the target color range.
